(19) United States
(12) Patent Application Publication
(10) Pub. No.: US 2013/0210786 A1
(43) Pub. Date: Aug. 15, 2013

(54) TREATMENT OF L-DOPA, DOPAMINE AGONIST AND/OR DOPAMINE ENHANCER INDUCED DISORDERS

(71) Applicant: Patrick Alexander Howson, Huntingdon (GB)

(72) Inventor: Patrick Alexander Howson, Huntingdon (GB)

(21) Appl. No.: 13/811,344

(22) PCT Filed: Jul. 20, 2011
(86) PCT No.: PCT/GB2011/051376

§ 371 Date: Apr. 5, 2013

Related U.S. Application Data
(60) Provisional application No. 61/366,015, filed on Jul. 20, 2010.

(51) Int. Cl. A61K 31/58; A61K 31/138; A61K 45/06

(52) U.S. Cl. CPC A61K 31/58; A61K 45/06; A61K 31/138
USPC 514/173; 540/17

(57) ABSTRACT

One or more agent selected from A/B-cis furostane, furostene, spirostane and spirostene steroidal sapogenins and ester, ether, ketone or glycosylated forms thereof, including E and/or F ring opened derivatives thereof, is used to treat or prevent L-DOPA, dopamine agonist and/or dopamine enhancer induced disorders, such as L-DOPA induced dyskinesia (LID), which is a side effect of L-DOPA, dopamine agonist and/or dopamine enhancer therapies, e.g., for Parkinson's disease. The agent according to the invention may be administered in association with the therapeutic agent for the treatment of the Parkinson's disease or another dopamine-responsive disorder.

### BACKGROUND OF THE INVENTION

The present invention relates to the treatment of disorders induced by use of L-DOPA, dopamine agonists, dopamine enhancers or any combination thereof.

### FIELD OF THE INVENTION

L-DOPA (L-3,4-dihydroxyphenylalanine; levodopa), dopamine agonists (including partial agonists) or dopamine enhancers are valuable agents in the treatment of disorders of dopamine deficiency and other dopamine-responsive disorders, of which Parkinson's disease and other parkinsonism disorders are the best known and most widely studied, although others include restless leg syndrome, dopamine-responsive dystonia (DRD), also known as hereditary progressive dystonia with diurnal fluctuation, Segawa's disease or Segawa's dystonia.

L-DOPA is a bioprecursor for dopamine, to which it is converted by the patient's metabolic processes.

L-DOPA is usually administered in association with a DOPA decarboxylase inhibitor which prevents the L-DOPA being converted to dopamine in the periphery. The DOPA decarboxylase cannot cross the blood-brain barrier, therefore, in the CNS the L-DOPA is metabolised to dopamine. Dopamine enhancers include substances and mixtures which block the metabolism of dopamine and so enhance the level of endogenous dopamine in tissues and blood in comparison with untreated patients are and therefore they prolong the effects of both endogenous dopamine and exogenous dopamine (e.g. following L-DOPA administration). Examples of dopamine enhancers include catecholamine-O-methyltransferase (COMT) enzyme inhibitors including entacapone and tolcapone and monoamine oxidase-B (MAO-B) inhibitors such as selegiline and rasagiline.

Dopamine agonists are substances and mixtures which bind to and activate dopamine receptors and thus mimic the actions (including the side effects) of dopamine. The dopamine agonists bromocriptine, pergolide, pramipexole, ropinirole, piribedil, cabergoline, apomorphine and lisuride are moderately effective against Parkinson's Disease.

All these agents are associated with certain side effects which limit their utility. Such side effects include dyskinesia, hypotension, arrhythmias, nausea, disturbed respiration, sleep disorders (for example, somnolence, insomnia and vivid dreams), dopamine dysregulation syndrome, hallucinations, and neuropsychiatric problems such as risk-taking, gambling tendency, impulse control disorders, anxiety, disorientation and confusion, psychosis and any combination thereof. These side effects and others are generally considered to be related via an underlying mechanism of overstimulation of the patient's dopaminergic system.

### BRIEF DESCRIPTION OF THE INVENTION

The present invention is based on our surprising finding that a class of steroidal sapogenin and saponin agents, previously described for treatment of Parkinson's and other neurodegenerative disorders, has important utility also in the treatment of L-DOPA, dopamine agonist and/or dopamine enhancer induced disorders, particularly side effects of L-DOPA and dopamine agonist therapies, and including combination therapies in which the L-DOPA and/or the dopamine agonist(s) is/are used in conjunction with one or more dopamine enhancers and/or one or more other active agent.

Such L-DOPA, dopamine agonist and/or dopamine enhancer induced disorders include, for example, disorders of the central nervous system related to overstimulation of the dopaminergic system through the use of L-DOPA, dopamine agonists and/or dopamine enhancers. Such disorders include, for example, dyskinesia, hypotension, arrhythmias, nausea, disturbed respiration, sleep disorders (for example, somnolence, insomnia and vivid dreams), dopamine dysregulation syndrome, hallucinations, and neuropsychiatric problems such as risk-taking, gambling tendency, impulse control disorders, anxiety, disorientation and confusion, psychosis and any combination thereof. L-DOPA-induced dyskinesia is commonly referred to as LID.

According to a first aspect of the present invention, there is provided a method of treating or preventing L-DOPA, dopamine agonist and/or dopamine enhancer induced disorders in a subject in need thereof, comprising administering to the subject an effective amount of one or more agent selected from A/B-cis furostane, furostene, spirostane and spirostene steroidal sapogenins and ester, ether, ketone and glycosylated forms thereof.

According to a second aspect of the present invention, there is provided an agent selected from A/B-cis furostane, furostene, spirostane and spirostene steroidal sapogenins and ester, ether, ketone and glycosylated forms thereof, for use in a method of treating or preventing L-DOPA, dopamine agonist and/or dopamine enhancer induced disorders in a subject in need thereof.

According to a third aspect of the present invention, there is provided a composition comprising an active agent selected from A/B-cis furostane, furostene, spirostane and spirostene steroidal sapogenins and ester, ether, ketone and glycosylated forms thereof, for use in a method of treating or preventing L-DOPA, dopamine agonist and/or dopamine enhancer induced disorders in a subject in need thereof.

According to a fourth aspect of the present invention, there is provided the use of an agent selected from A/B-cis furostane, furostene, spirostane and spirostene steroidal sapogenins and ester, ether, ketone and glycosylated forms thereof, in the manufacture of a medicament for treating or preventing L-DOPA, dopamine agonist and/or dopamine enhancer induced disorders in a subject in need thereof.

The present invention may be used in conjunction with methods of treatment of any dopamine-responsive disorders, such as, for example, Parkinson's disease, other parkinsonism disorders, restless leg syndrome or dopamine-responsive dystonia (DRD), to treat subjects suffering from those disorders to alleviate the side effects of conventional treatments for those disorders as described above. The method according to the present invention may thus be a method in which the active agent is administered simultaneously with, or shortly time-spaced from, administration of one or more therapeutic agent for the treatment of a disorder of dopamine deficiency and other dopamine-responsive disorders in the subject. Examples of such therapeutic agents are discussed above, and also below in the section headed “Administration With Treatment of Dopamine-Responsive Disorders”. The agents for use in the present invention have been found to be neurotrophic factor (NF) modulators that induce self-regulated homeostasis of more than one NF, for example brain-derived neurotrophic factor (BDNF) and glial-derived neurotrophic factor (GDNF). The agents for use in the present invention have been found to have no adverse side-effects and to be readily delivered to organs and tissues in need of treatment. The agents have been found to cross the blood-brain barrier. See, for example, PCT Patent Application No. PCT/GB2010/050098 and the publications referenced therein, which are incorporated herein by reference. A combination of the sapogenin agent and L-DOPA, or a dopamine agonist or enhancer may be used in the treatment of Parkinson's disease or any of the other diseases termed dopamine responsive disorders. The adjunct therapy with the combination of agents may be beneficial over the monotherapy with either of the agents individually, which may be due to the sapogenin lowering the side effects of overstimulation of the dopaminergic system. The combination therapy may be supplied simultaneously as a composition of the two agents, or may be supplied separately. The sapogenin may be supplied prior to the L-DOPA, or dopamine agonist or enhancer. The sapogenin may be smilagenin or sarsaspogenin or an analogue thereof. An aspect of the invention is the treatment of Parkinson's disease with a combination of smilagenin or sarsasapogenin and L-DOPA or a dopamine agonist or enhancer.

The agents for use in the present invention are also known from published patent and non-patent literature to have activity against a range of medical and non-medical physiological conditions. For example, smilagenin and its derivatives have been identified as valuable therapeutic agents in human and veterinary medicine and in non-therapeutic human and non-human animal treatments. See, for example, U.S. Pat. No. 3,890,438 (use of smilagenin and certain 4-substituted phenoxyisobutyric acid compounds against high blood cholesterol levels); U.S. Pat. No. 4,680,289 (use of smilagenin against obesity and diabetes obesity syndromes); U.S. Pat. No. 6,258,386 (use of smilagenin against cognitive dysfunction and allied conditions); WO-A-01/23406, WO-A-01/23407, WO-A-01/23408, and WO-A-01/49703 (use of smilagenin derivatives against cognitive dysfunction and allied conditions); and WO-A-02/079221 and WO-A-03/082893 (use of smilagenin and derivatives thereof against non-cognitive neurodegeneration, non-cognitive neuromuscular degeneration, motor-sensory neurodegeneration and loss of receptor function in the absence of cognitive, neural or neuromuscular impairment). Sarsasapogenin and its derivatives have been identified as valuable therapeutic agents in human and veterinary medicine and in non-therapeutic human and non-human animal treatments. See, for example, U.S. Pat. No. 4,680,289 (use of sarsasapogenin against obesity and diabetes obesity syndromes); Yi et al, Synthesis and Applications of Isotopically Labelled Compounds, 315 to 320, 1997 (Ed. J R Heys and D G Melillo) (use of sarsasapogenin against senile dementia); WO-A-99/48507 (use of sarsasapogenin against conditions characterised by a deficiency in membrane-bound receptor number or function); WO-A-01/23406 and WO-A-01/49703 (use of sarsasapogenin derivatives against cognitive dysfunction and allied conditions, including non-therapeutic use to enhance cognitive function in mentally healthy humans and animals); and WO-A-02/079221 and WO-A-03/082893 (use of sarsasapogenin and derivatives thereof against non-cognitive neurodegeneration, non-cognitive neuromuscular degeneration, motor-sensory neurodegeneration and loss of receptor function in the absence of cognitive, neural or neuromuscular impairment). The contents of these publications are incorporated herein by reference.

Therefore, the present invention may be used in conjunction with methods of treatment of humans and non-human animals using the medical and non-medical treatments (including prophylaxis) described and claimed in PCT Patent Application No. PCT/GB2010/050098 and/or in any of the prior publications identified in the preceding paragraph, whether individually or in any combination.

In accordance with the invention, the agents may be administered systemically or locally, as their delivery to the sites of action is found to be generally good. In particular, but without limitation, oral, topical and parenteral (e.g. intravenous) administration routes are found to be suitable, as discussed in more detail below. The small molecular size of the active agents relative to peptide, including protein, agents makes delivery of the agents to brain and CNS sites substantially easier than in the case of large molecule peptides. Oral administration is possible and preferred using the agents of the present invention.

As described in PCT Patent Application No. PCT/GB2010/050098, the agents for use in the present invention have a remarkably low level of (ant)agonistic binding capacity for a range of hormonal and other receptors and no enzyme binding capacity across a range of enzymes. They are therefore suitable for use in conjunction with a wide range of medical and non-medical treatments (including prophylaxis) using other active agents. They are suitable for use on both male and female subjects. They are also very suitable for use on elderly or infirm patients, who may be more susceptible than younger patients to neurological and/or psychiatric disorders, which may be aggravated or induced by active agents having receptor and/or enzyme binding capacity.

As described in PCT Patent Application No. PCT/GB2010/050098, the agents for use in the present invention are able to induce self-regulated homeostasis of neurotrophic factors (NFs), for example BDNF and/or GDNF, by modulating the subject's native NFs in a non-toxic manner under homeostatic control. By only modulating NFs in abnormal (damaged) tissue they minimise the risk of perturbing healthy tissues, thus reducing the likelihood of side effects including neurological and/or psychiatric disorders. The agents therefore exhibit limited and manageable side effects.

The expression “A/B-cis furostane, furostene, spirostane and spirostene steroidal sapogenin”, and related expressions, used herein, includes all E and/or F ring opened derivatives, for example pseudosapogenin and dihydrospeudosapogenin forms of the said A/B-cis furostane, furostene, spirostane and spirostene steroidal sapogenins In the unsaturated (-ene) forms of the compounds, one or more double bond is present at locations which do not affect the A/B-cis motif. Glycosylated forms of sapogenins are commonly referred to as saponins.

### DETAILED DESCRIPTION OF THE INVENTION

### Introduction

The evidence presented in this application shows that smilagenin, an A/B-cis steroidal sapogenin, alleviates the effects of L-DOPA-induced dyskinesia (LID), and in particular they raise the threshold dose of L-DOPA at which LID is observed in primates and they reduce or eliminate the LID symptoms otherwise induced at any particular dose of L-DOPA (see Example 1). This effect is produced without reducing the therapeutic benefit of L-DOPA on the parkinsonian disability. One embodiment of the invention described herein is therefore the combination of an A/B-cis steroidal sapogenin and L-DOPA in the treatment of Parkinsons Disease. The use of the agents in combination is preferable to use of either of the agents individually.

In conjunction with the evidence from PCT Patent Application No. PCT/GB2010/050098 and the publications referenced therein showing the effects of smilagenin and the related active agents on the induction of NFs or NF-receptors and countering neurodegeneration and promoting neuroregeneration, it is predictable that the agents of the present invention, namely one or more agent selected from A/B-cis furostane, furostene, spirostane and spirostene steroidal sapogenins and ester, ether, ketone and glycosylated forms thereof, are effective to treat or prevent L-DOPA, dopamine agonist and/or dopamine enhancer induced disorders in a subject in need thereof.

The evidence presented in this application is combinable with the evidence contained in PCT Patent Application No. PCT/GB2010/050098 and the publications referenced therein to support use of the present invention in the circumstances and for the reasons described in that PCT application. The novel circumstances include the new uses described on pages 14 to 24 of PCT Patent Application No. PCT/GB2010/050098 and in more detail elsewhere in that application, the contents of which are incorporated herein by reference.

Any aspect of the present invention may be practised or used simultaneously with any one or more of the other aspects of the invention, and any example or preference stated for one aspect of the present invention shall apply equally to any other aspect of the invention.

### “Treating or Preventing”

The expression “treating or preventing” and analogous terms used herein refers to all forms of healthcare intended to remove or avoid the disorder or to relieve its symptoms, including preventive, curative and palliative care, as judged according to any of the tests available according to the prevailing medical and psychiatric practice. An intervention which aims with reasonable expectation to achieve a particular result but does not always do so is included within the expression “treating or preventing”. An intervention which succeeds in slowing or halting progression of a disorder is included within the expression “treating or preventing”.

“Susceptible to”

The expression “susceptible to” and analogous terms used herein refers particularly to individuals at a higher than normal risk of developing a medical, health, wellbeing or psychiatric disorder, or a personality change, as assessed using the known risk factors for the individual or disorder. Such individuals may, for example, be categorised as having a substantial risk of developing one or more particular disorders or personality changes, to the extent that medication would be prescribed and/or special dietary, lifestyle or similar recommendations would be made to that individual.

### Toxicity and Side Effects

The agents according to the present invention have limited and manageable side effects and are non-toxic or essentially non-toxic in use.

In the context of pharmaceutical (including veterinary) use, this implies physiological acceptability of the agents, so that, within the scope of sound medical and veterinary judgement, the agents are suitable for use at an effective dosage in contact with cells of humans, mammals and other animals without undue toxicity, irritation, allergic response, undesirable side effects, and that such adverse events as may occur are deemed excessive or cannot be managed by side treatment, commensurate with a reasonable benefit/risk ratio.

In the context of functional foods, particularly foodstuffs, food supplements (including dietary supplements), beverages and beverage supplements, as well as topical preparations such as functional cosmetics and dermatological and other skin-contacting or eye-contacting preparations, this implies a corresponding assessment of benefit/risk and side effects, appropriate to the safety and toxicity standards for the particular composition or preparation and the particular use for which it is supplied.

### “Non-Therapeutic Method”

The known uses of the agents according to the present invention (see PCT Patent Application No. PCT/GB2010/050098 and the prior publications of known uses of the agents, discussed above) include non-therapeutic uses, for example, non-therapeutic uses to improve neurological or psychological functioning, or general health and wellbeing of an individual, non-therapeutic use to improve skin, bone, eye, muscle and other tissue health, and non-therapeutic use to assist recovery of muscle and tissues from exercise, exertion or wasting, improving endurance and reducing the feeling of fatigue (see PCT Patent Application No. PCT/GB2010/050098, paragraph bridging pages 14 and 15 and associated discussion).

In addition, the uses of the agents in accordance with the present invention can include non-therapeutic uses to improve the depth and quality of sleep, reduce vivid dreaming, nightmares and hallucinations, and to moderate behavioural or psychological problems associated with risk-taking or gambling behaviour associated with treatments using L-DOPA, dopamine agonists and/or dopamine enhancers.

A non-therapeutic use is generally characterised by a human subject's elective self-administration, typically oral, of a physiologically active agent in a composition without medical supervision. Typically, the intended benefits from this will be wellbeing or general health benefits in relation to conditions or perceived conditions that are (i) formally undiagnosed, (ii) undiagnosable according to clinical practice, or (iii) within the normal ranges of the healthy population and therefore not considered as disorders.

A non-therapeutic use can also be characterised by the absence of medical intervention or assistance at the stage of the subject's purchasing or acquiring the composition.

Still further, a non-therapeutic use can be characterised by the absence of medical claims by the supplier of the composition, so that the self-administration is not driven by a specific intention to treat a diagnosed disorder.

In addition to the examples of psychological functions given above that are treatable according to the non-therapeutic methods of the present invention, mild forms of psychiatric disorders associated with treatments using L-DOPA, dopamine agonists and/or dopamine enhancers, that are non-diagnosable according to clinical practice because the associated behaviours or thoughts do not cause significant distress to the individual or are not disruptive of his or her everyday functioning, may also be considered as conditions treatable non-therapeutically according to the present invention.

### Subjects

L-DOPA, dopamine agonist and/or dopamine enhancer induced disorders occur primarily in humans. Therefore, the subject of the treatment underlying the present invention is typically a human, particularly but not exclusively a human over the age of about 50 years.

However, the present invention may be practiced in a range of mammals, especially laboratory mammals which can also be affected by L-DOPA, dopamine agonist and/or dopamine enhancer induced disorders. Such mammals include non-human primates (e.g. apes, monkeys and lemurs), rabbits or rodents (e.g. rats, mice, hamsters, gerbils or guinea pigs), particularly such laboratory mammals as are used in the study of dopamine-responsive disorders, such as, for example, Parkinson's disease, other parkinsonism disorders or dopamine-responsive dystonia (DRD), and the present invention may be used to alleviate the side effects of the treatments under test on such mammal subjects.

### Agents

The active agents used herein may generally, but not essentially, have a molecular weight less than about 800, for example less than about 700, for example less than about 600, for example less than about 500, for example less than about 450.

Following the standard nomenclature from steroid chemistry, the left hand 6-membered ring is named the A ring and the adjacent ring to the A-ring is named the B-ring. Again following standard nomenclature from steroid chemistry, the carbon atoms are numbered so that the line of fusion between the rings occurs between the 5- and 10-position carbon atoms.

In A/B-cis steroidal furostane/ene or spirostane/ene sapogenins, the substituent or hydrogen atom at both the 5- and the 10-position carbon atoms are orientated 13 to (above) the plane of the molecule.

This has the effect of kinking the plane of the molecule to create a pharmacophore group which looks as follows in a three-dimensional drawing. The substituent or hydrogen atom at the 10-position carbon atom is labelled as “a” in the drawing and the substituent or hydrogen atom at the 5-position carbon atom is labelled as “b”; the C ring is only partially shown:

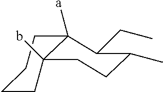

This is the A/B-cis motif.

Examples of A/B-cis furostane/ene and spirostane/ene sapogenins and their derivative forms disclosed in WO-A-99/48482, WO-A-99/48507, WO-A-01/23407, WO-A-01/23408, WO-A-02/079221, WO-A-03/082893, WO-A-2005/105825 and WO-A-2006/048665 may be particularly mentioned as active agents for use in the present invention. The particular sets of compounds, and individual compounds, disclosed in these publications, representative of the class of compounds which is the A/B-cis furostane/ene and spirostane/ene sapogenins and ester, ether, ketone and glycosylated forms thereof, and all E and/or F ring opened derivatives thereof, are incorporated herein by reference.

The ester, ether, ketone and glycoslyated forms of the A/B-cis furostane/ene and spirostane/ene sapogenins and their E and/or F ring opened derivatives may be such that one or more ester, ether, ketone and glycoslyated group may be present in the molecule. Generally speaking, an ester, ether, ketone or glycoslyated group may be formed at any one or more OH moiety of the A/B-cis sapogenin, using conventional chemical synthetic methods.

Examples of the active agents according to the present invention are the A/B-cis compounds represented by formula I in WO-A-01/23406 (see pages 6 to 11 of the published PCT application), formula II in WO-A-01/23406 (see pages 6 to 11 of the published PCT application), formula I in WO-A-01/23407 (see pages 6 to 11 of the published PCT application), formula II in WO-A-01/23407 (see pages 6 to 11 of the published PCT application), formula I in WO-A-01/23408 (see pages 6 to 10 of the published PCT application), formulae I, II and III in WO-A-01/49703 (see pages 7 to 15 of the published PCT application), formula II in WO-A-02/079221 (see pages 6 to 9 of the published PCT application), formula I in WO-A-03/082893 (see pages 3 to 17 of the published PCT application), formula Ia of WO-A-03/082893 (see pages 3 to 17 of the published PCT application), formula II in WO-A-03/082893 (see pages 3 to 17 of the published PCT application), formula III in WO-A-03/082893 (see pages 3 to 17 of the published PCT application), formula I in EP-A-1024146 (see pages 3 to 10 of the published EP application), and formula II in EP-A-102416 (see pages 3 to 10 of the published EP application). These examples are all specifically incorporated herein by reference.

For example, the molecules sarsasapogenin and smilagenin and their corresponding ester, ether, ketone and saponin (glycosylated) derivatives are useful active agents for the present invention. The compound timosaponin BII, which is an A/B-cis furostane saponin, is a useful active agent for the present invention.

Other useful active agents for the present invention include episarsasapogenin, epismilagenin, metagenin, samogenin, diotigenin, isodiotigenin, texogenin, yonogenin, mexogenin and markogenin and their corresponding ester, ether, ketone and saponin derivatives.

The active agent may be used in any suitable crystalline or amorphous form, and in any suitable anhydrous, hydrated or solvated form. Further details of such forms of sarsasapogenin and smilagenin and their derivatives are given in WO-A-2005/105825 and WO-A-2006/048665, to which specific reference is directed.

The esters may especially include 3-position esters such as the carboxylate (e.g. cathylate (ethoxycarbonyloxy), acetate, succinate, cinnamate, ferulate, propionate, butyrate, isobutyrate, valerate, isovalerate, caproate, isocaproate, diethylacetate, octanoate, decanoate, laurate, myristate, palmitate, stearate, benzoate, phenylacetate, phenylpropionate, cinnamate, p-nitrobenzoyloxy, 3,5-dinitrobenzoyloxy, p-chlorobenzoyloxy, 2,4-dichlorobenzoyloxy, p-bromobenzoyloxy, m-bromobenzoyloxy, p-methoxybenzoyloxy, phthalyl, glycinate, alaninate, valinate, phenylalaninate, isoleucinate, methioninate, argininate, asparaginate, aspartate, cysteinate, glutamate, histidinate, lysinate, prolinate, serinate, threoninate, tryptophanate, tyrosinate, fumarate, maleate), phosphonate and sulphonate esters.

The ethers may especially include 3-position ethers such as the alkoxy derivatives (e.g. methoxy, ethoxy, n-propoxy, s-propoxy, n-butoxy, s-butoxy, t-butoxy).

The ketones (sapogenones) are typically the 3-keto derivatives of the corresponding sapogenins, although other keto derivatives formed at different OH-bearing carbon atoms of the ring system are also possible. Examples of 3-keto sapogenones include sarsasapogenone, smilagenone, episarsasapogenone and epismilagenone.

Examples of suitable saponin compounds include the compounds in which the carbon atom at the 3-position (i.e. the carbon to which R₃ is attached) carries in place of R₃ an O-sugar moiety, for example a mono-, di- or tri-saccharide or higher polysaccharide or an acylated form thereof. Examples of such sugar groups include sugar groups selected from glucose, mannose, fructose, galactose, maltose, cellobiose, sucrose, rhamnose, xylose, arabinose, fucose, quinovose, apiose, lactose, galactose-glucose, glucose-arabinose, fucose-glucose, rhamnose-glucose, glucose-glucose-glucose, glucose-rhamnose, mannose-glucose, glucose-(rhamnose)-glucose, glucose-(rhamnose)-rhamnose, glucose-(glucose)-glucose, galactose-(rhamnose)-galactose and acylated (e.g. acetylated) derivatives thereof.

Pseudosapo(ge)nins are ring-opened derivatives of the respective spirostane/ene sapogenins or saponins in which the F ring is opened and locked. Pseudosapo(ge)nins may have saturation or unsaturation at the C20-C22 bond. The saturated form is sometimes referred to as a “dihydropseudosapo(ge)nin” form.

The active agents for the present invention may be used singly or in any desired combination.

Administration with Treatment of Dopamine-Responsive Disorders

The agents and compositions of the present invention may suitably be administered at the same time as, or shortly before, or shortly after (or in any desired combination of these options), agents for the treatment of disorders of dopamine deficiency and other dopamine-responsive disorders in a subject in need thereof. Such disorders include, for example, Parkinson's disease, other parkinsonism disorders, restless leg syndrome and DRD.

Agents for the treatment of disorders of dopamine deficiency, other dopamine-responsive disorders and dopamine/dopamine agonist-induced disorders include, for example, dopamine precursors, dopamine prodrugs, dopamine agonists and partial agonists, dopa decarboxylase inhibitors, COMT inhibitors, MAO-B inhibitors, anticholinergics, adamantanes, calcium channel agonists, adenosine alpha-2 receptor antagonists, glucagon-like peptide-1 mimetics, glutamate release inhibitors, metabotropic glutamate receptor 5 negative allosteric modulators, metabotropic glutamate receptor 5 (mGluR5) antagonists, selective serotonin reuptake inhibitors (SSRIs), monoamine reuptake inhibitors, antioxidants, N-methyl-D-aspartate (NMDA) receptor antagonists, benzothiazoles and n-NOS inhibitors such as, for example, levodopa, docarpamine, tripeptide 1 (GHK or Gly-His-Lys), PRX1, apomorphine, bromocriptine, cabergoline, lisuride, pergolide, pramipexole, ropinirole, rotigotine, pardoprunox, aplindore (DAB452), PRX5, carbidopa, entacapone, tolcapone, selegiline, rasagiline, safinamide, trihexyphenidyl, benztropine, ethopropazine, amantadine, isradipine, istradefylline, fipamezole (JP-1730), vipadenant (BIIB014 or V2006), LuAA4707, preladenant (SCH 420814), exendin-4, FP0011, ADX48621, ADX10059, AFQ056, clavulanic acid, citalopram, escitalopram, fluoxetine, paroxetine, sertraline, vanoxerine, atomoxetine, duloxetine, amineptine, bupropion, tesofensine, hyperforin, coenzyme Q10, vitamin E, creatinine, memantine, riluzole, PRX2; and any combination thereof.

The therapeutic agents for the treatment of disorders of dopamine deficiency and other dopamine-responsive disorders, for example, those mentioned above, may be administered individually or in any desired combination. The examples given to illustrate the above classes of therapeutic agents include derivative or modified forms thereof. The agents and compositions of the present invention, when administered in association with therapeutic agents for the treatment of disorders of dopamine deficiency and other dopamine-responsive disorders, may be administered individually or in any desired combination.

The combined therapies of—on the one hand—treatment of disorders of dopamine deficiency and other dopamine-responsive disorders and—on the other hand—treatment of L-DOPA, dopamine agonist and/or dopamine enhancer induced disorders, may be performed by simultaneous administration of the desired therapeutic agents or compositions. In one example, the active agents used in the present invention may be administered in admixture with the agents for treatment of disorders of dopamine deficiency and other dopamine-responsive disorders, in which case the agents and co-agents or co-ingredients will be provided in the same composition. Alternatively or additionally, some or all desired active agents for treatment of disorders of dopamine deficiency and other dopamine-responsive disorders may be administered separately from the agents according to the present invention, either simultaneously or in a time-spaced manner, in which case the agents for treatment of disorders of dopamine deficiency and other dopamine-responsive disorders will be provided in a first composition or set (kit) of compositions and the agents according to the present invention will be provided in a second composition or set (kit) of compositions, preferably with instructions for the administration protocol to be followed.

All such combined compositions, sets and kits are aspects of the present invention to the extent that they are associated with the active agents, methods, uses and compositions according to the present invention as defined and claimed herein.

Suitable combined compositions may include any of the agents of the present invention, when administered in association with therapeutic agents for the treatment of disorders of dopamine deficiency and other dopamine-responsive disorders. Specific compositions may include L-Dopa, one of the dopamine agonists bromocriptine, pergolide, pramipexole, ropinirole, piribedil, cabergoline, apomorphine or lisuride or one of the dopamine enhancers carbidopa, entacapone, tolcapone, selegiline, rasagiline, safinamide combined with a sapogenin analogue, for example smilagenin or sarsapogenin. Compositions may include combinations of smilagenin and L-Dopa or Sarsasapogenin and L-Dopa. Such combinations may be formulated for medicinal use, and may be used as pharmaceuticals.

### Other Co-Agents or Co-Ingredients

The compositions of the present invention may, if desired, include one or more co-agents and/or one or more co-ingredients, other than agents for the treatment of disorders of dopamine deficiency and other dopamine-responsive disorders, as described in more detail below in connection with the compositions and administration routes.

In particular, metabolic adjuvants, compounds that increase ketone body levels (ketogenic compounds), the tricarboxylic acid (TCA) cycle intermediates, compounds that are convertible in vivo to TCA intermediates, energy-enhancing compounds, or any mixture thereof may be used as co-agents or co-ingredients in the compositions of the present invention.

Some or all desired co-agents or co-ingredients may be administered in admixture with the agents according to the present invention, in which case the agents and co-agents or co-ingredients will be provided in the same composition. Alternatively or additionally, some or all desired co-agents or co-ingredients may be administered separately from the agents according to the present invention, either simultaneously or in a time-spaced manner, in which case the agents will be provided in a first composition or set (kit) of compositions and the co-agents or co-ingredients will be provided in a second composition or set (kit) of compositions, preferably with instructions for the administration protocol to be followed.

Metabolic adjuvants include vitamins (e.g. Vitamin E), minerals, antioxidants and other related compounds (for example, ascorbic acid, biotin, calcitriol, cobalamin, folic acid, niacin, pantothenic acid, pyridoxine, retinol, retinal (retinaldehyde), retinoic acid, riboflavin, thiamine, α-tocopherol, phytylmenaquinone, multiprenylmenaquinone, calcium, magnesium, sodium, aluminium, zinc, potassium, chromium, vanadium, selenium, phosphorus, manganese, iron, fluorine, copper, cobalt, molybdenum, iodine, or any combination thereof.

Ketogenic compounds generally enhance endogenous fat metabolism (oxidation) by the recipient and thereby raise the blood ketone levels, and include for example C₃₋₈ ketones such as acetone, D-β-hydroxybutyrate, metabolic precursors of D-β-hydroxybutyrate (for example acetoacetyl precursors such as acetoacetyl-1,3-butanediol, acetoacetyl-D-β-hydroxybutyrate and acetoacetylglycerol; esters such as esters of D-β-hydroxybutyrate with monohydric, dihydric or trihydric alcohols; or polyesters of D-β-hydroxybutyrate such as poly-D-(3-hydroxybutyrate or terminally oxidised poly-D-β-hydroxybutyrate having from about 2 to about 100 repeats, e.g. from about 3 to about 10 repeats), metabolic precursors of acetoacetate, or any combination thereof.

TCA intermediates include citric acid, aconitic acid, isocitric acid, α-ketoglutaric acid, succinic acid, fumaric acid, malic acid, oxoacetic acid, or any combination thereof.

Compounds that are convertible in vivo to TCA intermediates include 2-keto-hydroxypropanol, 2,4-dihydroxybutanol, 2-keto-4-hydroxybutanol, 2,4-dihydroxybutyric acid, 2-keto-4-hydroxybutyric acid, aspartates, mono- and di-alkyl-oxaloacetates, pyruvate, glucose-6-phosphate, or any combination thereof.

Energy-enhancing compounds include, for example, Coenzyme CoQ-10, creatine, creatine derivatives, L-carnitine, n-acetyl-carnitine, L-carnitine derivatives, or any combination thereof. These compounds enhance energy production by a variety of means. Carnitine will increase the metabolism of fatty acids. CoQ-10 serves as an electron carrier during electron transport within the mitochondira. Accordingly, the addition of such compounds with active agents such as medium chain triglycerides (MCTs) will increase metabolic efficiency, especially in individuals who may be nutritionally deprived.

The co-agent, when present, may be provided in the form of a metabolic precursor such as a complex with one or more cations or as a salt, for use in therapy or nutrition. Examples of cations and typical physiological salts include sodium, potassium, magnesium, calcium salts, in each case the cation being balanced by a physiological counterion forming a salt complex such as L-lysine, L-arginine, methyl glucamine or others known in the art. The preparation and use of such metabolic precursors is described in WO-A-98/41201 and WO-A-00/15216, the disclosures of which are incorporated herein by reference.

### Compositions and Administration Routes

The active agent may be administered in the form of a composition comprising the active agent and any suitable additional component. The composition may, for example, be a pharmaceutical composition (medicament), a foodstuff, food supplement or beverage. Such a composition may contain a mixture of the specified compounds, and/or of their physiologically acceptable esters, amides, salts, solvates, analogs, or other suitable derivatives. In general, reference herein to the presence of one active agent and/or other component of a composition includes within its scope the presence of a mixture of two or more of such agents and/or components.

The pharmaceutical composition can be administered by any appropriate route including, but not limited to, oral, nasogastric, rectal, transdermal, parenteral (e.g. subcutaneous, intramuscular, intravenous, intramedullary and intradermal injections or infusions), intranasal, transmucosal, implantation, vaginal, topical, buccal and sublingual.

It is a typical feature of the use of a small-molecule somewhat lipophilic agent—as many of the active agents are—that the administration site can be remote from the brain of the mammal to be treated, the agent migrating through the bloodstream and crossing the blood-brain and/or blood-nerve barriers.

The term “pharmaceutical composition” in the context of this invention means a composition comprising an active agent and comprising additionally pharmaceutically acceptable carriers, diluents, adjuvants, excipients, or vehicles, such as preserving agents, fillers, disintegrating agents, buffering agents, preserving agents, penetration enhancers, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. Suitable dosage forms include, for example, tablets, dragees, powders, elixirs, syrups, liquid preparations, including suspensions, sprays, inhalants, tablets, lozenges, emulsions, solutions, granules, capsules and suppositories, as well as liquid preparations for injections, including liposome preparations. Techniques and formulations generally may be found in Remington, Pharmaceutical Sciences, Mack Publishing Co., Easton, Pa., latest edition.

The terms “foodstuff”, “food supplement”, “beverage” and “beverage supplement” used herein have the normal meanings for those terms, and are not restricted to pharmaceutical preparations. These compositions are adapted for oral ingestion. Supplement compositions (e.g., a food supplement or beverage supplement) are arranged to be added to foods and beverages and ingested with them. A foodstuff typically may include calorific materials such as fats, oils and carbohydrates, as well as proteins and sources of minerals and fibre. Examples of compositions include dairy, cereal, vegetable, meat, fish, poultry or fruit based foodstuffs. Examples of beverages include carbonated and uncarbonated beverages, fruit juices, infusion drinks such as coffee or teas, for example herbal tea, fruit tea, Japanese green tea or Indian or Chinese tea. Compositions may comprise milk or milk-derived components, such as powdered milk and/or lactose and/or casein. The milk or milk-derived components are preferably derived from cows or goats. Plant-derived milks such as soya milk may be used. An edible composition may comprise one or more fermented components. The composition may comprise yogurt. Food supplements may, for example, contain vitamins, minerals, caffeine, ephedra alkaloids.

For further details of the compositions and administration routes usable in the present invention, please refer to PCT Patent Application No. PCT/GB2010/050098 (pages 39 to 59) and the publications referenced therein, the contents of all which are incorporated herein by reference.

In each case, the composition may suitably contain one or more other active agents, which may be selected from the A/B-cis spirostane or spirostene steroidal sapogenins and ester, ether, ketone and glycosylated forms thereof, including E and/or F ring opened derivatives thereof, other sapo(ge)nins, other non-sapo(ge)nin active agents, or any combination thereof. The composition may contain one or more biologically inert ingredients, for example diluents, carriers and excipients, which serve purposes related to presentation, administration or delivery of the physiologically active component, or which provide associated benefits to the subject separately from the physiological effects of the active component. The carriers may comprise plant materials such as soya protein. The composition may, for example, also comprise any one or more of preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms.

The composition for use in the present invention, particularly the pharmaceutical composition, may be in unit dosage form, whereby a certain number of such forms are administered to the subject in a certain time period, according to the condition to be treated or prevented. Alternatively, the composition may be in bulk form, whereby a certain weight or volume of the bulk composition is measured out and administered to the subject in a certain time period, according to the condition to be treated or prevented.

However, toxicity is not considered to be a problem with these active agents, even at the higher dosages. The selection of appropriate dosages is thus within the ability of one of ordinary skill in this art, without undue burden. The administered daily dosage of the active is preferably between about 0.1 and about 35 mg/kg body weight, e.g. between about 1 and about 25 mg/kg body weight, preferably administered as one full dose or two half-doses per day. For adult human use, the daily dosage may conveniently be between about 10 and about 2500 mg per day.

The composition for use in the present invention may suitably contain other therapeutic and/or non-therapeutic bioactive agents, as discussed above.

For further details of suitable composition forms and dosages, and examples of conditions and diseases treatable in conjunction with the present invention, please refer to WO-A-99/48482, WO-A-99/48507, WO-A-01/23407, WO-A-01/23408, WO-A-02/079221, WO-A-03/082893, WO-A-2005/105825 and WO-A-2006/048665.

The active agents are suitably formulated with one or more carrier, excipient and/or diluent in the composition. Generally speaking, any conventional carrier, excipient and/or diluent used for pharmaceutical compositions, oral compositions such as foodstuffs, food supplements and beverages, or topical compositions such as cosmetic, eye or skin preparations may be used.

Many of the active agents are relatively lipophilic, and in this case solubilising and/or suspending and/or dispersing agents may suitably be used to maintain the active agent in solution or suspension or dispersion in the composition.

Two group of solubilising and/or suspending and/or dispersing agents that may particularly be mentioned are the MCTs and the medium chain fatty acids (MCFAs). These are lipophilic compounds having fatty acid chains with chain lengths of between about 4 and about 12 carbon atoms.

Preferred examples of MCTs are represented by the following general formula (I):

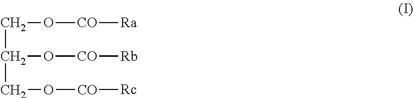

wherein Ra, Rb and Rc are, independently of each other, selected from saturated or unsaturated fatty acid residues having 4 to 12 carbon atoms in the carbon backbone.

Preferred examples of MCFAs are represented by the following general formula (II):

HO—CO-Rd (II)

wherein Rd is a saturated or unsaturated fatty acid residue having from 4 to 12 carbon atoms in the carbon backbone.

Examples of Ra, Rb, Re and Rd include residues of caproic (C6:0), caprylic (C8:0), capric (C10:0) and lauric (C12:0) acids. In the standard naming system, the number immediately after the letter C indicates the carbon chain length and the number immediately after the colon (:) indicates the number of unsaturated bonds. Such MCTs and MCFAs can be obtained in known manner from natural sources such as coconut oil, palm kernel oil and camphor drupes (fruits). The residues of one or more than one fatty acids may be present in a commercial MCT or MCFA product.

MCTs for use in the present invention may, for example, be selected from tri-C6:0 MCT, tri-C8:0 MCT and tri-C10:0 MCT.

As previously mentioned, some or all desired co-agents or co-ingredients, including co-agents for treatment of disorders of dopamine deficiency and other dopamine-responsive disorders, may be administered in admixture with the agents according to the present invention, in which case the agents and co-agents or co-ingredients will be provided in the same composition. Alternatively or additionally, some or all desired co-agents or co-ingredients, including co-agents for treatment of disorders of dopamine deficiency and other dopamine-responsive disorders, may be administered separately from the agents according to the present invention, either simultaneously or in a time-spaced manner, in which case the agents will be provided in a first composition or set (kit) of compositions and the co-agents or co-ingredients will be provided in a second composition or set (kit) of compositions, preferably with instructions for the administration protocol to be followed. Any suitable administration protocol may be followed, and this may include periodically repeated dosages of the agents and any co-agents or co-ingredients, in any desired order or sequence and at the same or different dosages at each administration.

### INDUSTRIAL APPLICABILITY AND UTILITY

The present invention makes available a valuable new treatment for certain debilitating side effects of L-DOPA, dopamine agonist and/or dopamine enhancer based therapies, including LID which is a side effect of therapies for treating Parkinson's disease, other parkinsonism disorders, restless leg syndrome or dopamine-responsive dystonia (DRD).

The agents for use in the treatments are small molecules, and not peptides (e.g. proteins), which supports the potential utility of the present invention outside the elite clinical setting, where elaborate delivery apparatus for administration of peptide active agents directly into the brain or CNS may be unavailable.

Since many patients suffering from Parkinson's disease, other parkinsonism disorders, restless leg syndrome or DRD may be relatively old, frail, or in rather poor general health, they are often susceptible to other medical, neurological or psychiatric disorders. Often it is not predictable with any certainty which of a range of other disorders or conditions will arise. The agents for use in the present invention show a remarkably low level of (ant)agonistic binding capacity for a range of hormonal and other receptors and no enzyme binding capacity across a range of enzymes. They are therefore suitable for use in conjunction with a wide range of medical and non-medical treatments (including prophylaxis) using other active agents. Prior to the present invention, such other disorders or conditions, or the individual's susceptibility to them, often contraindicated the treatment of L-DOPA, dopamine agonist and/or dopamine enhancer induced side effects, including LID, in the context of Parkinson's disease patients, as very often the treatment would carry a substantial risk of promoting other disorders, problems or conditions in such a patient. Therefore, the utility of the present invention in treating the disorders and conditions in ways that are easier and simpler than before, and which are applicable to a wider group of patients in this way, represents a substantial advance in medical science and healthcare practice in these important areas of human health.

The invention may be described using the following statements:

1. A method of treating or preventing L-DOPA, dopamine agonist and/or dopamine enhancer induced disorders in a subject in need thereof, comprising administering to the subject an effective amount of one or more agent selected from A/B-cis furostane, furostene, spirostane and spirostene steroidal sapogenins and ester, ether, ketone and glycosylated forms thereof, including E and/or F ring opened derivatives thereof.

2. A method according to statement 1, wherein the L-DOPA, dopamine agonist and/or dopamine enhancer induced disorder is selected from disorders of the central nervous system related to overstimulation of the dopaminergic system through the use of L-DOPA, dopamine agonists and/or dopamine enhancers.

3. A method according to statement 1 or statement 2, wherein the L-DOPA, dopamine agonist and/or dopamine enhancer induced disorder is selected from dyskinesia, hypotension, arrhythmias, nausea, disturbed respiration, sleep disorders (for example, somnolence, insomnia and vivid dreams), dopamine dysregulation syndrome, hallucinations, and neuropsychiatric problems such as risk-taking, gambling tendency, impulse control disorders, anxiety, disorientation and confusion, psychosis and any combination thereof.

4. A method according to any one of the preceding statements, wherein the L-DOPA, dopamine agonist and/or dopamine enhancer induced disorder is L-DOPA-induced dyskinesia and the subject is a human undergoing L-DOPA, dopamine agonist and/or dopamine enhancer treatment for Parkinson's disease, other parkinsonism conditions, restless leg syndrome or dopamine-responsive dystonia (DRD).

5. A method according to any of the preceding statements, wherein the method is used in conjunction with non-therapeutic methods for the treatment or prevention of neurological or psychiatric conditions that are within the normal range of a population and/or are not diagnosable disorders.

6. A method according to any one of the preceding statements, wherein the method is used in circumstances without clinical control of the administration protocol to the subject.

7. A method according to any one of the preceding statements, wherein the active agent is selected from sarsasapogenin, smilagenin, episarsasapogenin, epismilagenin, timosaponin BII, metagenin, samogenin, diotigenin, isodiotigenin, texogenin, yonogenin, mexogenin and markogenin, their corresponding ester, ether, ketone and saponin (glycosylated) derivatives, and E and/or F ring opened derivatives thereof.

8. A method according to any one of the preceding statements, wherein the active agent is selected from sarsasapogenin and smilagenin, their corresponding ester, ether, ketone and saponin (glycosylated) derivatives, and E and/or F ring opened derivatives thereof.

9. A method according to any one of the preceding statements, wherein the active agent is administered in association with administration of one or more therapeutic agent for the treatment of a disorder of dopamine deficiency or another dopamine-responsive disorder in the subject.

10. A method according to statement 9, wherein the active agent, to be administered in association with administration of one or more therapeutic agent for the treatment of a disorder of dopamine deficiency or another dopamine-responsive disorder in the subject, comprises sarsasapogenin.

11. A method according to statement 9, wherein the active agent, to be administered in association with administration of one or more therapeutic agent for the treatment of a disorder of dopamine deficiency or another dopamine-responsive disorder in the subject, comprises smilagenin.

12. A method according to statement 9, 10 or 11, wherein the one or more therapeutic agent for the treatment of a disorder of dopamine deficiency and other dopamine-responsive disorder in the subject is selected from dopamine precursors such as, for example, levodopa and carbidopa; dopamine prodrugs such as, for example, docarpamine, tripeptide 1 (GHK or Gly-His-Lys) and PRX1; dopamine agonists and partial agonists such as, for example, apomorphine, bromocriptine, cabergoline, lisuride, pergolide, pramipexole, ropinirole, rotigotine, pardoprunox. Aplindore (DAB452) and PRX5; COMT inhibitors such as, for example, entacapone, tolcapone; MAO-B inhibitors such as, for example, selegiline, rasagiline and safinamide; anticholinergics such as, for example, trihexyphenidyl, benztropine and ethopropazine; adamantanes such as, for example, amantadine; calcium channel agonists such as, for example, isradipine; adenosine alpha-2 receptor antagonists such as, for example, istradefylline, fipamezole (JP-1730), vipadenant (BIIB014 or V2006), LuAA4707 and preladenant (SCH 420814); glucagon-like peptide-1 mimetics such as, for example, exendin-4; glutamate release inhibitors such as, for example, FP0011; metabotropic glutamate receptor 5 negative allosteric modulators (mGluR5 NAMs) such as, for example, ADX48621 and ADX10059; metabotropic glutamate receptor 5 (mGluR5) antagonists such as, for example, AFQ056; selective serotonin reuptake inhibitors (SSRIs) such as, for example, seradaxin; antioxidants such as, for example, coenzyme Q10, vitamin E and creatinine; N-methyl-D-aspartate (NMDA) receptor antagonists such as, for example, memantine; benzothiazoles such as, for example, riluzole; n-NOS inhibitors such as, for example, PRX2; and any combination thereof.

13. A method according to any one of the preceding statements, wherein the one or more active agent is used in conjunction with one or more co-agent selected from metabolic adjuvants, compounds that increase ketone body levels (ketogenic compounds), the tricarboxylic acid (TCA) cycle intermediates, compounds that are convertible in vivo to TCA intermediates, energy-enhancing compounds, and any mixture thereof.

14. A method according to any one of the preceding statements, wherein the one or more active agent is administered in a composition comprising the active agent and any suitable additional component, for example, a pharmaceutical composition (medicament), a foodstuff, food supplement or beverage (e.g. a carbonated beverage), or a topical composition such as a cosmetic, eye or skin (e.g. dermatological) composition.

15. A method according to statement 14, wherein the one or more active agent is present in the composition with one or more solubilising and/or suspending and/or dispersing agents to maintain the active agent in solution or suspension or dispersion in the composition, for example medium chain triglycerides (MCTs) or medium chain fatty acids (MCFAs).

16. A method according to any one of the preceding statements, wherein the administration takes place by a route selected from oral, nasogastric, rectal, transdermal, parenteral (e.g. subcutaneous, intramuscular, intravenous, intramedullary and intradermal injections or infusions), intranasal, transmucosal, implantation, vaginal, topical, buccal and sublingual.

17. A method according to any one of the preceding statements, wherein the subject is a human.

18. A method according to any one of the preceding statements, wherein the administration takes place by mouth and the subject is a human.

19. An agent selected from A/B-cis furostane, furostene, spirostane and spirostene steroidal sapogenins, and ester, ether, ketone and glycosylated forms thereof, including E and/or F ring opened derivatives thereof, for use in a method of treating or preventing L-DOPA, dopamine agonist and/or dopamine enhancer induced disorders in a subject by administering to the subject an effective amount of one or more such agent.

20. An agent according to statement 19, for use in a method as defined in any one of statements 2 to 18.

21. A composition comprising one or more active agent selected from A/B-cis furostane, furostene, spirostane and spirostene steroidal sapogenins, and ester, ether, ketone and glycosylated forms thereof, including E and/or F ring opened derivatives thereof, for use in a method of treating or preventing L-DOPA, dopamine agonist and/or dopamine enhancer induced disorders in a subject by administering to the subject an effective amount of one or more such agent in the said composition.

22. A composition according to statement 21, for use in a method as defined in any one of statements 2 to 18.

23. Use of one or more agent selected from A/B-cis furostane, furostene, spirostane and spirostene steroidal sapogenins, and ester, ether, ketone and glycosylated forms thereof, including E and/or F ring opened derivatives thereof, in the manufacture of a medicament for treating or preventing L-DOPA, dopamine agonist and/or dopamine enhancer induced disorders in a subject.

24. A use according to statement 23, wherein the medicament is for use in a method as defined in any one of statements 2 to 18.

### EXAMPLE

In the following Example, these abbreviations are used: h=hours; min=minutes; s=seconds; s.c.=sub-cutaneous; p.o.=per oro (by mouth); w/v=weight/volume; v/v=volume/volume; b.i.d.=bis in die (twice a day); s.e.=standard error.

The following Example shows that smilagenin reduces L-DOPA induced dyskinesia (LID) in MPTP-lesioned macaques.

Ten female cynomolgus monkeys (macaques) (Macaca fascicularis, 3.0-4.5 kg, 4-6 years old) were acclimatised to the experimental setting and procedures for 3 months and baseline behaviour was assessed in all animals. The macaques received MPTP (0.2 mg/kg/day, s.c.) until moderate-marked, stable, parkinsonian symptoms developed.

Damage caused by the neurotoxin MPP⁺, a metabolite of MPTP, mimics the degeneration of nigrostriatal dopaminergic neurones observed in neurodegenerative diseases such as Parkinson's disease (Mytinlineou et al, Science, 225, 529-531 (1984)). The most prominent biochemical changes induced by this toxin include increased levels of dopamine and its metabolites in the substantia nigra pars compacta and in the caudate nucleus (Burns et al, Proc. Natl. Acad. Sci. USA, 80, 4546-4550 (1983)) and a reduction in dopamine uptake in nigrostriatal synaptosomal preparations (Heikkila et al, J. Neurochem., 44, 310-313 (1985)).

Following stabilisation of parkinsonian disability, the macaques were administered L-DOPA (Madopar, 20 mg/kg b.i.d., p.o.) for 19 weeks before being administered L-DOPA+vehicle (hydroxypropylmethylcellulose, HPMC, 0.5% w/v containing Tween 80, 0.2% v/v, group 1, n=5) or L-DOPA+smilagenin (20 mg/kg/day, p.o., group 2, n=5) for a further 18 weeks. Smilagenin was then washed out for 10 weeks and all the macaques received only L-DOPA twice daily over this 10 week period.

Following the 10 week washout, the macaques were challenged with L-DOPA (6, 12, 20, 30 or 40 mg/kg, p.o.) or vehicle and the level of dyskinesia over the subsequent 6 h period was assessed by a neurologist blinded to treatment using a monkey dyskinesia rating scale. On the days before being challenged with L-DOPA or vehicle the macaques did not receive the second L-DOPA treatment of the day (i.e. the last L-DOPA administration that the macaques received was 24 h before the L-DOPA or vehicle challenge). Each macaque received a challenge once every 3 days and received every challenge (vehicle and L-DOPA, 6, 12, 20, 30 or 40 mg/kg) in a randomised order over the course of the experiment. On the days between challenges, the macaques received L-DOPA (20 mg/kg, b.i.d., p.o.) as normal.

When challenged with vehicle or L-DOPA (6 mg/kg), no dyskinesia was observed over the subsequent 6 h in either group. In group 1, following challenge with L-DOPA (12, 20, 30 or 40 mg/kg), the macaques exhibited dose-dependent dyskinesia up to the 30 mg/kg dose. The level of dyskinesia exhibited following 40 mg/kg of L-DOPA was similar to the level induced by 30 mg/kg of L-DOPA. In group 2, no dyskinesia was observed following challenge with L-DOPA (6 or 12 mg/kg). Dyskinesia was observed following challenge with L-DOPA at 20, 30 or 40 mg/kg, however, there was less dyskinesia compared to group 1 at each of these doses (Table 1). Across all doses of L-DOPA, the smilagenin-treated macaques exhibited significantly less dyskinesia (53% reduction) than vehicle-treated macaques (p=0.0085) and this effect was most pronounced following the L-DOPA (30 mg/kg) challenge (76% reduction, p<0.0001).

TABLE 1
Summary of LID in vehicle and smilagenin treated macaques. Macaques
were treated with smilagenin for 18 weeks and followed by a 10 week
washout of smilagenin before being challenged with L-DOPA

| | | | | | | | | | % | |
|---|---|---|---|---|---|---|---|---|---|---|
| | | | | | | | | | reduction in | |
| | | | | | | | | | LID | Significance |
| | | | | | | | | | compared | compared to |
| | L- | Number of LID events in each stated time | | | | | | | to corresponding | corresponding |
| | DOPA | period (hours) post challenge | | | | | | | vehicle | vehicle |
| Group | (mg/kg) | 0-1 | 1-2 | 2-3 | 3-4 | 4-5 | 5-6 | Total | group | group |
| Vehicle | 0 | 0 | 0 | 0 | 0 | 0 | 0 | 0 | — | — |
| Vehicle | 6 | 0 | 0 | 0 | 0 | 0 | 0 | 0 | — | — |
| Vehicle | 12 | 5 | 4 | 0 | 0 | 0 | 0 | 9 | — | — |
| Vehicle | 20 | 30 | 31 | 24 | 4 | 0 | 0 | 89 | — | — |
| Vehicle | 30 | 25 | 94 | 53 | 6 | 0 | 0 | 178 | — | — |
| Vehicle | 40 | 38 | 65 | 54 | 16 | 0 | 0 | 173 | — | — |
| Total | — | 98 | 194 | 131 | 26 | 0 | 0 | 449 | — | — |
| Smilagenin | 0 | 0 | 0 | 0 | 0 | 0 | 0 | 0 | 0 | n.c |
| Smilagenin | 6 | 0 | 0 | 0 | 0 | 0 | 0 | 0 | 0 | n.c |
| Smilagenin | 12 | 0 | 0 | 0 | 0 | 0 | 0 | 0 | 100 | p = 0.0417 |
| Smilagenin | 20 | 8 | 29 | 6 | 0 | 0 | 0 | 43 | 52 | n.s |
| Smilagenin | 30 | 3 | 29 | 11 | 0 | 0 | 0 | 43 | 76 | p < 0.0001 |
| Smilagenin | 40 | 8 | 56 | 48 | 14 | 0 | 0 | 126 | 27 | n.s |
| Total | — | 19 | 114 | 65 | 14 | 0 | 0 | 212 | 53 | p = 0.0085 |

*= Analysis was performed using a two-way ANOVA with group and time as factors.
n.c = not calculable due to no dyskinesia, n.s = not significant (p > 0.05).

The data collected from each 6 h observation were further analysed with respect to on-time (defined as the time that the macaques did not exhibit bradykinesia). L-DOPA increased the amount of on-time in both groups and the total on-time was not significantly different (p>0.05) in both groups (Table 2).

TABLE 2
Summary of on-time in vehicle and smilagenin treated
macaques. Macaques were treated with smilagenin for
18 weeks and followed by a 10 week washout of smilagenin
before being challenged with L-DOPA

| | | | Percent on-time |
|---|---|---|---|
| | L-DOPA | Minutes spent in on | normalised to 0 mg/kg |
| | dose | time (mean ± s.e. | L-DOPA (mean ± s.e. |
| Group | (mg/kg) | mean) | mean) |
| Vehicle | 0 | 130 ± 57 | 100 |
| Vehicle | 6 | 182 ± 72 | 140 ± 40 |
| Vehicle | 12 | 210 ± 61 | 162 ± 29 |
| Vehicle | 20 | 198 ± 67 | 152 ± 34 |
| Vehicle | 30 | 238 ± 47 | 183 ± 20 |
| Vehicle | 40 | 240 ± 50 | 185 ± 21 |
| Smilagenin | 0 | 114 ± 43 | 100 |
| Smilagenin | 6 | 134 ± 43 | 118 ± 32 |
| Smilagenin | 12 | 126 ± 35 | 111 ± 28 |
| Smilagenin | 20 | 178 ± 29 | 156 ± 16 |
| Smilagenin | 30 | 224 ± 13 | 196 ± 6 |
| Smilagenin | 40 | 230 ± 24 | 202 ± 10 |

Analysis was performed using a two-way ANOVA with group and L-DOPA dose as factors. There was no significant difference (p>0.05) in on-time between the two groups.

The MPTP-lesioned and L-DOPA treated macaques used in this experiment provide an accepted model for L-DOPA-induced dyskinesia.

The foregoing broadly describes the present invention, without limitation. Variations and modifications as will be readily apparent to those of ordinary skill in this art are intended to be included in the scope of this application and any resultant patent.

1. A method of treating or preventing L-DOPA, dopamine agonist and/or dopamine enhancer induced disorders in a subject in need thereof, comprising administering to the subject an effective amount of one or more agent selected from A/B-cis furostane, furostene, spirostane and spirostene steroidal sapogenins and ester, ether, ketone and glycosylated forms thereof, including E and/or F ring opened derivatives thereof, wherein the L-DOPA, dopamine agonist and/or dopamine enhancer induced disorder is selected from dyskinesia, hypotension, arrhythmias, nausea, disturbed respiration, sleep disorders (for example, somnolence, insomnia and vivid dreams), dopamine dysregulation syndrome, hallucinations, and neuropsychiatric problems such as risk-taking, gambling tendency, impulse control disorders, anxiety, disorientation and confusion, psychosis and any combination thereof.

2. (canceled)

3. The method according to claim 1, wherein the L-DOPA, dopamine agonist and/or dopamine enhancer induced disorder is dyskinesia.

4. The method according to claim 1, wherein the L-DOPA, dopamine agonist and/or dopamine enhancer induced disorder is L-DOPA-induced dyskinesia and the subject is a human undergoing L-DOPA, dopamine agonist and/or dopamine enhancer treatment for Parkinson's disease, other parkinsonism conditions, restless leg syndrome or dopamine-responsive dystonia (DRD).

5. The method according to claim 1, wherein the method is used in conjunction with non-therapeutic methods for the treatment or prevention of neurological or psychiatric conditions that are within the normal range of a population and/or are not diagnosable disorders.

6. The method according to claim 1, wherein the method is used in circumstances without clinical control of the administration protocol to the subject.

7. The method according to claim 1, wherein the active agent is selected from sarsasapogenin, smilagenin, episarsasapogenin, epismilagenin, timosaponin BII, metagenin, samogenin, diotigenin, isodiotigenin, texogenin, yonogenin, mexogenin and markogenin, their corresponding ester, ether, ketone and saponin (glycosylated) derivatives, and E and/or F ring opened derivatives thereof.

8. The method according to claim 1, wherein the active agent is selected from sarsasapogenin and smilagenin, their corresponding ester, ether, ketone and saponin (glycosylated) derivatives, and E and/or F ring opened derivatives thereof.

9. The method according to claim 1, wherein the active agent is administered in association with administration of one or more therapeutic agent for the treatment of a disorder of dopamine deficiency or another dopamine-responsive disorder in the subject, wherein the one or more therapeutic agent for the treatment of a disorder of dopamine deficiency and other dopamine-responsive disorder in the subject is selected from dopamine precursors, dopamine prodrugs, dopamine agonists and partial agonists, dopa decarboxylase inhibitors, COMT inhibitors, MAO-B inhibitors, anticholinergics, adamantanes, calcium channel agonists, adenosine alpha-2 receptor antagonists, glucagon-like peptide-1 mimetics, glutamate release inhibitors, metabotropic glutamate receptor 5 negative allosteric modulators, metabotropic glutamate receptor 5 (mGluR5) antagonists, selective serotonin reuptake inhibitors (SSRIs), monoamine reuptake inhibitors, antioxidants, N-methyl-D-aspartate (NMDA) receptor antagonists, benzothiazoles and n-NOS inhibitors such as, for example, levodopa, docarpamine, tripeptide 1 (GHK or Gly-His-Lys), PRX1, apomorphine, bromocriptine, cabergoline, lisuride, pergolide, pramipexole, ropinirole, rotigotine, pardoprunox, aplindore (DAB452), PRX5, carbidopa, entacapone, tolcapone, selegiline, rasagiline, safinamide, trihexyphenidyl, benztropine, ethopropazine, amantadine, isradipine, istradefylline, fipamezole (JP-1730), vipadenant (BIIB014 or V2006), LuAA4707, preladenant (SCH 420814), exendin-4, FP0011, ADX48621, ADX10059, AFQ056, clavulanic acid, citalopram, escitalopram, fluoxetine, paroxetine, sertraline, vanoxerine, atomoxetine, duloxetine, amineptine, bupropion, tesofensine, hyperforin, coenzyme Q10, vitamin E, creatinine, memantine, riluzole, PRX2, and any combination thereof.

10. The method according to claim 1, wherein the active agent is sarsasapogenin.

11. The method according to claim 1, wherein the active agent is smilagenin.

12. (canceled)

13. The method according to claim 1, wherein the one or more active agent is used in conjunction with one or more co-agent selected from metabolic adjuvants, compounds that increase ketone body levels (ketogenic compounds), the tricarboxylic acid (TCA) cycle intermediates, compounds that are convertible in vivo to TCA intermediates, energy-enhancing compounds, and any mixture thereof.

14. The method according to claim 1, wherein the one or more active agent is administered in a composition comprising the active agent and any suitable additional component, for example, a pharmaceutical composition (medicament), a foodstuff, food supplement or beverage (e.g. a carbonated beverage), or a topical composition such as a cosmetic, eye or skin (e.g. dermatological) composition.

15. The method according to claim 13, wherein the one or more active agent is present with one or more solubilising and/or suspending and/or dispersing agents to maintain the active agent in solution or suspension or dispersion, for example medium chain triglycerides (MCTs) or medium chain fatty acids (MCFAs).

16. The method according to claim 1, wherein the administration takes place by a route selected from oral, nasogastric, rectal, transdermal, parenteral (e.g. subcutaneous, intramuscular, intravenous, intramedullary and intradermal injections or infusions), intranasal, transmucosal, implantation, vaginal, topical, buccal and sublingual.

17-24. (canceled)

25. A composition comprising one or more active agent selected from A/B-cis furostane, furostene, spirostane and spirostene steroidal sapogenins, and ester, ether, ketone and glycosylated forms thereof, including E and/or F ring opened derivatives thereof; and L-DOPA, a dopamine agonist and/or a dopamine enhancer.

26. The composition according to claim 25 comprising L-DOPA and an active agent selected from sarsasapogenin and smilagenin, their corresponding ester, ether, ketone and saponin (glycosylated) derivatives, and E and/or F ring opened derivatives thereof.

27. A method of treating Parkinson's disease using a combination of L-DOPA, a dopamine agonist and/or a dopamine enhancer and one or more active agent selected from A/B-cis furostane, furostene, spirostane and spirostene steroidal sapogenins, and ester, ether, ketone and glycosylated forms thereof, including E and/or F ring opened derivatives thereof.

28. The method of claim 27 wherein the combination is supplied simultaneously.

29. The method of claim 27 wherein the active agent is supplied prior to the L-DOPA, dopamine agonist and/or a dopamine enhancer.

30. The method of claim 27 wherein treatment is carried out using L-DOPA and smilagenin or sarsaspogenin.
